(19) United States
(12) Patent Application Publication
(10) Pub. No.: US 2024/0105286 A1
(43) Pub. Date: Mar. 28, 2024

(54) SYSTEM AND METHOD FOR QUANTIFICATION OF SYNERGISTIC EFFECTS OF MULTI-COMBINATION COMPOUNDS

(71) Applicant: CytoSolve, Inc., Cambridge, MA (US)

(72) Inventors: V.A. Shiva AYYADURAI, Belmont, MA (US); Prabhakar DEONIKAR, Grosse Pointe Woods, MI (US)

(73) Assignee: CytoSolve, Inc., Cambridge, MA (US)

(21) Appl. No.: 18/474,019

(22) Filed: Sep. 25, 2023

Related U.S. Application Data
(60) Provisional application No. 63/376,981, filed on Sep. 23, 2022.

(51) Int. Cl. G16C 20/30

(52) U.S. Cl. CPC G16C 20/30

(57) ABSTRACT

A process and a system are disclosed for determining if a combination of a set of chemical compounds has either a synergistic, additive, or antagonistic effect on a disease, e.g., osteoarthritis, or a biological function, e.g., oxidative stress. In one step of the process, for a particular ensemble of molecular pathway models representing the disease or biological function along with the particular set of biomarkers for that disease or biological function, computation is performed to quantify for a particular set of chemical compounds, their effects individually, sequentially, and in combination on the biomarkers. In another step of the process, comparison of these effects is performed to determine if the combinations of the chemical compounds behave synergistically, additively, or antagonistically. The end results are combinations of chemical compounds along with their dosage levels, which synergistically affect disease or biological function of interest.

### CROSS-REFERENCE TO RELATED APPLICATIONS

This application claims priority to U.S. Provisional Application No. 63/376,981, which was filed on Sep. 23, 2022, and which is incorporated herein by reference for all purposes in its entirety.

### TECHNICAL FIELD

This disclosure pertains to the arts of molecular pathway modeling, computational systems biology, and development of multi-combination therapies for addressing diseases e.g., osteoarthritis or biological functions e.g., oxidative stress.

### BACKGROUND

There are nearly half-a-million chemical compounds, from natural or synthetic sources, that are in production and use. The range of possible compounds is estimated to be between 10¹⁸ to 10²⁰⁰. The 2-, 3-, . . . or n-combinations of these compounds are nearly infinite. Ancient and indigenous systems of medicine learned the “art” of combining combinations of compounds to affect specific diseases, e.g., osteoarthritis or biological functions, e.g., oxidative stress. Western and conventional medicine is recognizing the need for combinations of compounds to alleviate diseases and affect biological functions.

Systems provided in the art reveal that such combination therapies are derived at the bedside, in an opportunistic manner; by trial and error in the lab or clinic; and by brute force high throughput in vitro screening. The latter is limited at best to two-combination applications. The art further reveals that in the pharmaceutical industry, most drugs approved by the FDA are single compounds; there are several hundred that are two-combination drugs; a handful are three-combination drugs; and there is only one four-combination drug. In the nutraceutical and dietary supplement industry, most products are combinations of many chemical compounds. However, the formulations are derived in an ad hoc manner with little to no molecular systems understanding of how the combinations of those compounds interact with complex molecular pathways (systems of reactions) in vitro or in vivo.

### SUMMARY

The present disclosure herein provides a system, which can take as input: an individual set of compounds; an ensemble of molecular pathway models representing a disease or a biological function; and a set of biomarkers indicative of the disease or a biological function. Further, this present disclosure provides a system to determine if the 2-, 3-, . . . or n-combinations of compounds have an additive, antagonistic, or synergistic effect on the disease or a biological function. Finally, this present disclosure provides a system to quantify the synergistic effect and derive the specific dosage levels of each compound in the combination that elicit the synergistic effect.

In particular, in one embodiment, there is provided A method of determining particular combinations of compounds having a synergistic effect on a particular biological system of molecular mechanisms, the method comprising: (a) identifying a set of N compounds, each compound of the set of N compounds interacting with a biological system of molecular mechanisms, wherein N is an integer greater than or equal to 2; (b) determining one or more combinations of compounds, each combination of compounds consisting of two or more compounds of the set of N compounds; (c) determining, for each combination of the one or more combinations of compounds, a set of compound sequences, each compound sequence including all compounds in the combination and being different from other of the compound sequences in the set of compound sequences; (d) determining, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels; (e) determining a maximum level of the set of levels as a first level of the first biomarker and a minimum level of the set of levels as a second level of the first biomarker; (f) determining, using the mathematical model and for the particular combination, a third level of the first biomarker resulting from a simulated administration to the biological system of the compounds of the particular combination simultaneously using the first set of dosages; and (g) determining, based on the first level, the second level, and the third level, an effect of the particular combination on the first biomarker using the first set of dosages.

In another embodiment, there is provided An apparatus for determining particular combinations of compounds having a synergistic effect on a particular biological system of molecular mechanisms, the apparatus comprising: processing circuitry configured to (a) identify a set of N compounds, each compound of the set of N compounds interacting with a biological system of molecular mechanisms, wherein N is an integer greater than or equal to 2; (b) determine one or more combinations of compounds, each combination of compounds consisting of two or more compounds of the set of N compounds; (c) determine, for each combination of the one or more combinations of compounds, a set of compound sequences, each compound sequence including all compounds in the combination and being different from other of the compound sequences in the set of compound sequences; (d) determine, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels; (e) determine a maximum level of the set of levels as a first level of the first biomarker and a minimum level of the set of levels as a second level of the first biomarker; (f) determine, using the mathematical model and for the particular combination, a third level of the first biomarker resulting from a simulated administration to the biological system of the compounds of the particular combination simultaneously using the first set of dosages; and (g) determine, based on the first level, the second level, and the third level, an effect of the particular combination on the first biomarker using the first set of dosages.

Further, in another embodiment, there is provided A system for determining particular combinations of compounds having a synergistic effect on a particular biological system of molecular mechanisms, the system comprising: a biological system modeling device having first processing circuitry; a computer-readable memory; and a manufacturing device having second processing circuitry, wherein the first processing circuitry is configured to (a) identify a set of N compounds, each compound of the set of N compounds interacting with a biological system of molecular mechanisms, wherein N is an integer greater than or equal to 2; (b) determine one or more combinations of compounds, each combination of compounds consisting of two or more compounds of the set of N compounds; (c) determine, for each combination of the one or more combinations of compounds, a set of compound sequences, each compound sequence including all compounds in the combination and being different from other of the compound sequences in the set of compound sequences; (d) determine, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels; (e) determine a maximum level of the set of levels as a first level of the first biomarker and a minimum level of the set of levels as a second level of the first biomarker; (f) determine, using the mathematical model and for the particular combination, a third level of the first biomarker resulting from a simulated administration to the biological system of the compounds of the particular combination simultaneously using the first set of dosages; (g) determine, based on the first level, the second level, and the third level, an effect of the particular combination on the first biomarker using the first set of dosages; and (h) store, when determining that the effect of the particular combination on the first biomarker is synergistic, the particular combination and the first set of dosages in the computer-readable memory, and wherein the second processing circuitry is configured to access the particular combination and the first set of dosages stored in the computer-readable memory, and generate a product based on the particular combination and the first set of dosages.

### BRIEF DESCRIPTION OF DRAWINGS

For a more complete understanding, the following disclosure can be taken in conjunction with the figures wherein:

FIG. 1 represents the system for determining an effect of a combination of compounds and deriving dosage levels of each compound in a combination that elicits a synergistic effect;

FIG. 2 represents the user interface where the user can provide inputs to the system;

FIG. 3 represents the outputs from the Pathway Modeling Module;

FIG. 4A represents individual effect of apigenin on COX-2 production over simulations periods of 2 days;

FIG. 4B represents individual effect of apigenin on PGE2 production over simulations periods of 2 days;

FIG. 4C represents individual effect of apigenin on TRPV1 production over simulations periods of 2 days;

FIG. 4D represents individual effect of apigenin on CGRP production over simulations periods of 2 days;

FIG. 5A represents individual effect of hesperidin on COX-2 production over simulations periods of 2 days;

FIG. 5B represents individual effect of hesperidin on PGE2 production over simulations periods of 2 days;

FIG. 5C represents individual effect of hesperidin on TRPV1 production over simulations periods of 2 days;

FIG. 5D represents individual effect of hesperidin on CGRP production over simulations periods of 2 days;

FIG. 5E represents individual effect of hesperidin on ROS production over simulations periods of 2 days;

FIG. 6A represents combination effect of hesperidin on COX-2 production over simulations periods of 2 days;

FIG. 6B represents combination effect of hesperidin on PGE2 production over simulations periods of 2 days;

FIG. 6C represents combination effect of hesperidin on TRPV1 production over simulations periods of 2 days;

FIG. 6D represents combination effect of hesperidin on CGRP production over simulations periods of 2 days;

FIG. 6E represents combination effect of hesperidin on ROS production over simulations periods of 2 days;

FIG. 7 illustrates a type of computer usable with embodiments of the present disclosure; and

FIG. 8 illustrates another type of computer usable with embodiments of the present disclosure.

### DETAILED DESCRIPTION

Given the vast and growing number of chemical compounds and even greater number of potential combinations, and further complicated by the dosage ranges of each compound in a combination, there is a need for computing to discover optimal combinations that affect disease or biological function. Specifically, the systems and methods for discovering 2-, 3-, . . . or n-combinations of compounds requires first, the ability to mathematically model diseases and biological functions by integrating large number of individual molecular pathways using a Pathway Modeling Module. One example of such a Pathway Modeling Module is CytoSolve®, which can integrate large systems of molecular pathway models to scale to model complex diseases and biological functions. (Further details are described in Ayyadurai, V. A., & Dewey, C. F. (2011). CytoSolve: A Scalable Computational Method for Dynamic Integration of Multiple Molecular Pathway Models. Cellular and molecular bioengineering, 4(1), 28-45, which is incorporated herein by reference in its entirety). While CytoSolve allows modeling of complex diseases and biological functions, a system and method neither for identifying and quantifying the synergistic combinations of 2-, 3-, . . . or n-compounds nor for enabling a user to identify and manufacture such synergistic combinations exists.

Combinations of compounds may have an additive, antagonistic, or synergistic effect on a specific disease or biological function. Systems and methods to discover synergistic—unexpected, non-linear, non-obvious—effects can have enormous financial, intellectual, and medical benefits. For example, from an intellectual property standpoint, when it comes to patenting combination therapies, patent reviewers demand evidence of synergistic effects prior to granting a multi-combination therapy patent. Financially, many manufacturers of ad hoc combination therapies, particularly in the nutraceutical and dietary supplements field, will benefit from discovering the right combinations and dosages of their ingredients and compounds to elicit a particular effect. Today, it's not only unclear if many of these combinations are beneficial, but also if they are efficiently manufactured, i.e., putting too much of one compound that has no effect beyond a particular dosage can be inefficient. Medically, finding the right dose combination of those compounds can serve to reduce toxicity and increase efficacy, since a given combination at lower dosage of each compound may synergistically affect multiple molecular pathways to alleviate a particular disease, versus giving a very large dosage of one compound that only affects a single molecular pathway and may result in toxic outcome.

A combination of compounds is said to have an additive effect if the total beneficial effect, as measured on a single biomarker or a set of biomarkers, is equal to the sum of the individual beneficial effects of each compound on the same set of biomarkers. A combination of compounds is said to have an antagonistic effect if the total negative effect, as measured on a single biomarker or a set of biomarkers, is equal to the sum or multiple of the individual negative effects of each compound on the same set of biomarkers.

A combination of compounds is said to have a synergistic effect if the total beneficial effect, as measured on a single biomarker or a set of biomarkers, is not equal to the sum, but is rather a multiple, non-linear combination of the individual beneficial effects of each compound on the same set of biomarkers.

Therefore, there exists a need in the art for a system that can take as input: an individual set of compounds; an ensemble of molecular pathway models representing a disease or a biological function; specifics about the organism for which the combination may be valuable; and, a set of biomarkers indicative of the disease or a biological function.

Further, there exists a need in the art for a system, to determine if the 2-, 3-, . . . or n-combinations of compounds have an additive, antagonistic, or synergistic effect on the disease or a biological function.

Finally, there exists a need in the art for a system, to quantify the synergistic effect and derive the specific dosage levels of each compound in the combination that elicits the synergistic effect, that can then be manufactured.

The following disclosure, when taken in conjunction with the presented figures, sets forth a method and system to quantify the synergistic effects of combinations of compounds on biological functions.

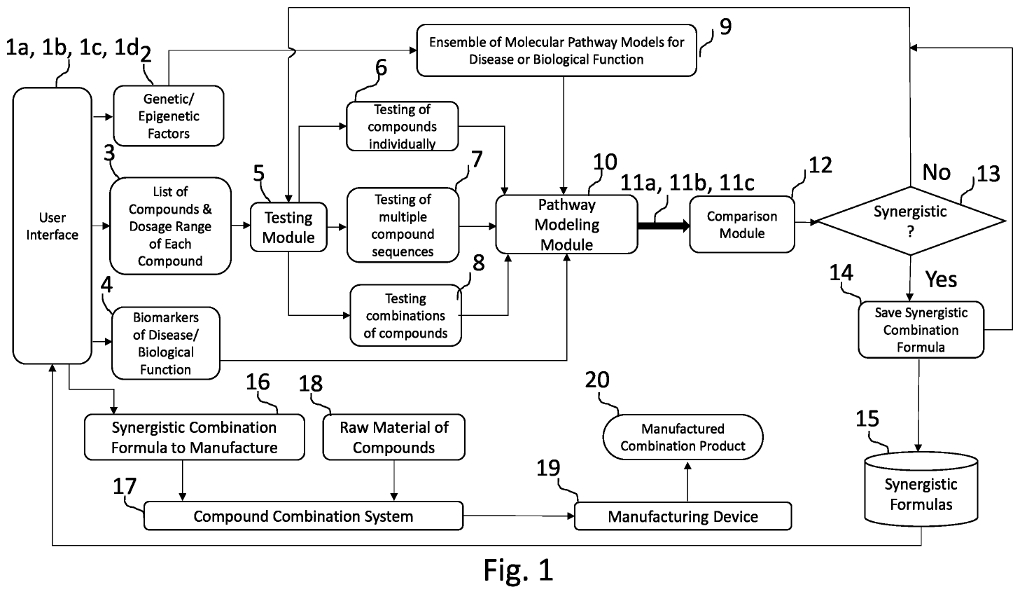

Turning to FIG. 1, many forms of inputs are received by the system that can affect a disease or a biological function.

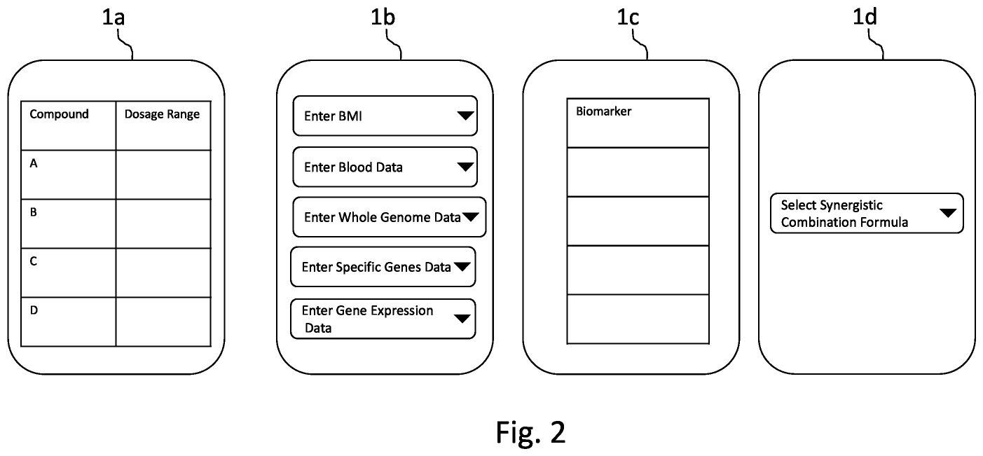

As shown in FIG. 2, a user interface 1 allows the user of the system to provide various inputs to the system. The first component 1a allows user to select the compounds and provide an upper and lower limit of dosages for the compounds The second component 1b allows the user to provide personalized data for an individual such as body mass index (BMI), blood test data, whole genome data, specific genes of interest for a disease or biological function for that individual, and gene expression data. The third component 1c of the user interface allows the user to choose a combination of compounds of interest and/or one or more biomarkers for that particular disease or biological function. The fourth component 1d allows the user to select a Synergistic Combination Formula that is selected for manufacturing.

Genetic/epigenetic factors 2, a list of compounds and dosage range of each compound 3, and a set of biomarkers of disease/biological function 4 are outputted based on the input received by the user interface, the input being based on or including the components 1a, 1b, 1c, 1d as discussed above.

The Genetic/Epigenetic Factors 2 are the inputs that are used by the Ensemble of Molecular Pathway Models for Disease/Biological Function 9 to set specific variables in the molecular pathway models. These variables may include molecular species concentrations and kinetic rate constants in the Ensemble of Molecular Pathway Models for Disease/Biological Function 9

The compounds and dosage ranges of each compound 1a entered in the User Interface 1 is provided as an input list of compounds and dosage ranges of each compound 3 to the Testing Module 5. The output of the Testing Module 5 contains experimental conditions for the testing of compounds individually 6; testing of multiple compound sequences 7; and testing of combination of compounds 8, for a particular dosage of each compound. These experimental conditions, along with ensemble of molecular pathway models for disease/biological function 9 and a set of biomarkers 4, where each biomarker of the set of biomarkers indicates the state of the disease or biological function are provided as an input to the Pathway Modeling Module 10.

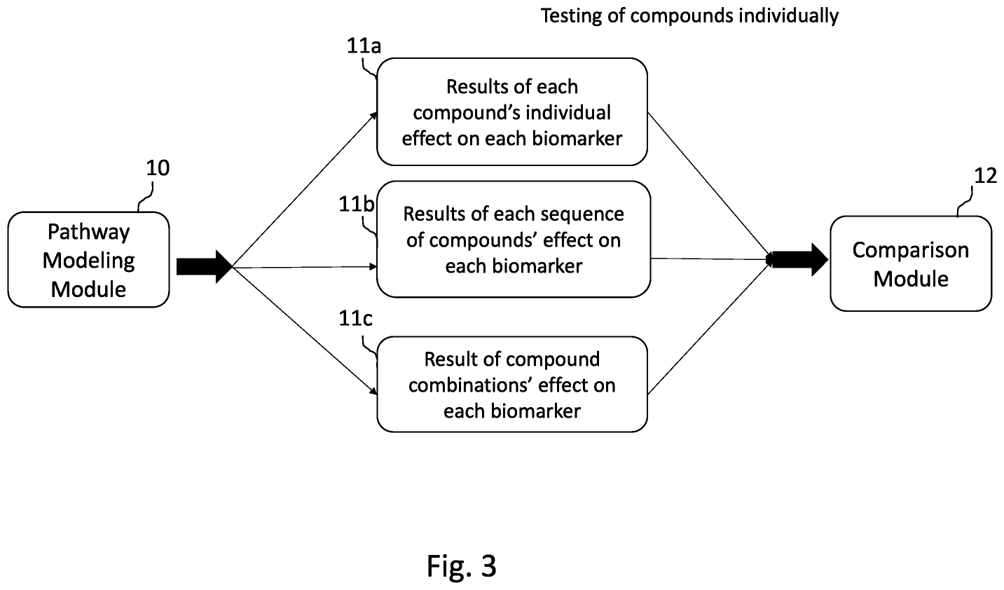

The molecular pathways are integrated by Pathway Modeling Module 10 to obtain predictive and quantitative effects of compounds measured in terms of the biomarkers 4 of the disease or a biological function. Three outputs are generated by the Pathway Modeling Module, as shown in FIG. 3: (1) the effect of set of individual compounds on biomarkers 11a; (2) the effect of sequential addition of individual compounds from a set of compounds on biomarkers 11b; and (3) the effect of combinations of compounds from a set of compounds on biomarkers 11c. These outputs from Pathway Modeling Module are provided as inputs to the Comparison Module 12.

The Comparison Module 12 determines if the 2-, 3-, . . . or n-combinations of compounds have an additive, antagonistic, or synergistic effect on the biomarkers associated with the disease or a biological function, and outputs the determination of the effect. To determine whether the specific combination acts synergistically, in an additive manner, or in an antagonistic manner to modulate the disease or biological function, it is necessary to quantify the additive effect range—two endpoints—for a particular biomarker. Determining the first endpoint of this range involves measuring or estimating a biomarker as a result of a sequential addition of compounds at their specific dosages. Determining the second endpoint of this range involves reversing the sequential addition of the compounds at their specific dosages and measuring or estimating the biomarker as a result. These two endpoints provide a lower and upper limit for the additive effect range of that biomarker for the specific dosages of these two compounds.

When n is greater than 2, there are more than two possible sequential additions of compounds. A biomarker can be measured or estimated as a result of each sequential addition of compounds at their specific dosages. The first endpoint of the range is the minimum biomarker level that is measured or estimated as a result of each sequential addition. The second endpoint of the range is the maximum biomarker level that is measured or estimated as a result of each sequential addition.

Once the additive effect range is determined, the compounds at the specific dosages are added in combination simultaneously to estimate the levels of the same biomarker. If the value of the biomarker obtained by the simultaneous addition of compounds lies within the additive effect range, then the combination of these two compounds is deemed to have an additive effect on that particular biomarker. If the value of biomarker obtained by the simultaneous addition of both compounds is not in the additive effect range, the two compounds may have a synergistic or an antagonistic effect. A combination is deemed to be synergistic if the value of a biomarker is outside of the additive effect range, e.g., lower or higher than the endpoints of the additive effect range, provided that the combination has a beneficial effect. The combination can be determined to have a beneficial effect if the level of the biomarker is lower or higher than a control level and is indicative of a positive effect on a biological function. The combination can be determined to be synergistic if the value of the biomarker obtained by the simultaneous addition of both compounds is different (e.g., higher or lower) than a value of the biomarker as a result of administration of each of compound in the combination individually and has a positive effect on the biological function.

The system of FIG. 1 determines at step 13 whether the output from Comparison Module 12 is a synergistic effect or not. If the output from Comparison Module 12 is a synergistic effect, then the system saves the synergistic combination formula 14 used to produce a synergistic effect in Synergistic Combination Formula repository 15, which contains a list of combination of compounds with their respective dosages that have synergistic effect on the biomarkers of the disease or a biological function. After the synergistic combination formula 14 is saved, the Testing Module 5 outputs the experimental conditions 6, 7, and 8 with a new set of dosages and iterates the process. Each dosage of a compound of the new set of dosages can be within the dosage range for the respective compound received by user interface 1. The dosages can be incremented by a dosage step size. The step size can be received as an input by the user interface 1. The repository of Synergistic Combination Formula 15 with their respective dosages is then made available by the system so that it can be accessed by the User interface 1.

If the output from the Comparison Module 12 is not a synergistic effect, then the Testing Module 5 repeats outputting the experimental conditions 6, 7, and 8 with a new set of dosages and iterates the process. Further, the above process can be performed for a single biomarker, and then repeated for other individual biomarkers for the same set of compounds.

Further, the user can select a synergistic combination formula to manufacture 16 from the repository of Synergistic Combination Formula provided by user interface 1, and the synergistic combination formula to manufacture 16 is further provided as an input to Compound Combination System 17, which selects the raw material of the compounds 18 and instructs and controls the manufacturing device 19 to make the manufactured combination product 20.

As an example, a study was performed using the System to identify a combination of compounds that has synergistic effect on a disease such as joint pain. (Further details are provided by Ayyadurai VAS, Deonikar P. In Silico Modeling and Quantification of Synergistic Effects of Multi-Combination Compounds: Case Study of the Attenuation of Joint Pain Using a Combination of Phytonutrients. Applied Sciences. 2022; 12(19):10013, which is incorporated herein by reference in its entirety).

As represented in FIG. 1, the inputs to the Pathway Modeling Module 10 of the System are: individual set of compounds 3—apigenin and hesperidin—that affect joint pain, the effect on joint pain when tested one individual compound at a time 6; the effect on joint pain when tested by sequential addition of apigenin followed by hesperidin, and hesperidin followed by apigenin 7; combinations of apigenin and hesperidin that affect joint pain and the effect on joint pain when tested as a combination 8; an ensemble of molecular pathway models 9 for joint pain including arachidonic acid metabolism, PGE2 signaling, COX-2 synthesis, and oxidative stress; and a set of five biomarkers 4 Prostaglandin E2 (PGE-2), transient receptor potential cation channel subfamily V member 1 (TRPV1), calcitonin gene-related peptide (CGRP), cyclooxygenase-2 (COX-2), and reactive oxygen species (ROS), where each biomarker indicates the state of the disease or biological function. Reducing the levels of the biomarkers of osteoarthritis leads to beneficial effects.

These molecular pathway models are integrated by Pathway Modeling Module 10 in FIG. 1 to obtain predictive and quantitative effects of apigenin and hesperidin measured in terms of the biomarkers of joint pain.

Individual Compound Outputs

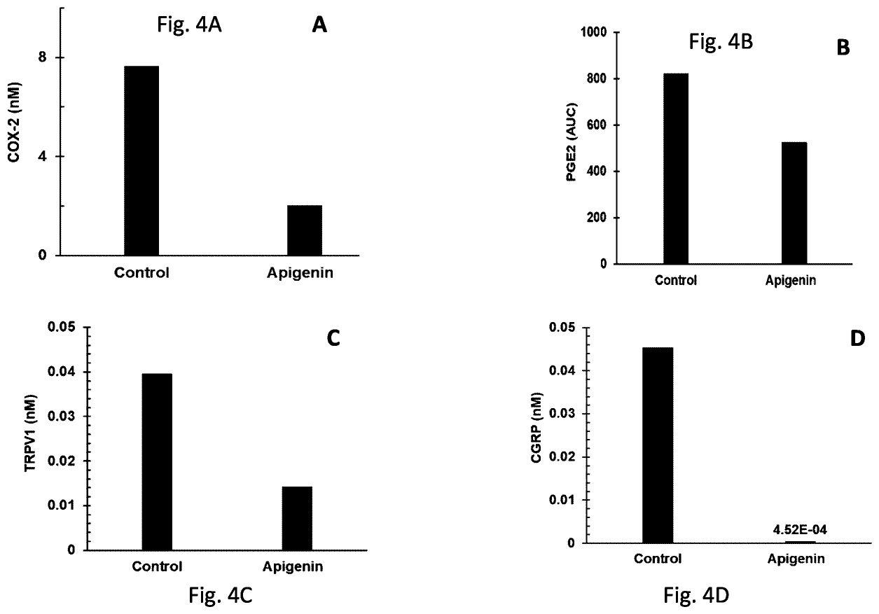

One set of outputs from Pathway Modeling Module 10 in FIG. 1 for the individual compound apigenin 6 is its effect on the five biomarkers of joint pain 11a, as in FIG. 3. When the input to the Pathway Modeling Module 10 was individual compound apigenin, it lowered the biomarker COX-2 compared to control as shown in FIG. 4A. When the input to the Pathway Modeling Module 10 was individual compound apigenin, it lowered the biomarker PGE2 compared to control as shown in FIG. 4B. When the input to the Pathway Modeling Module 10 was individual compound apigenin, it lowered the biomarker TRPV1 compared to control as shown in FIG. 4C. When the input to the Pathway Modeling Module 10 was individual compound apigenin, it lowered the biomarker CGRP compared to control as shown in FIG. 4D. When the input to the Pathway Modeling Module 10 was individual compound apigenin, it had no effect on the biomarker ROS compared to control.

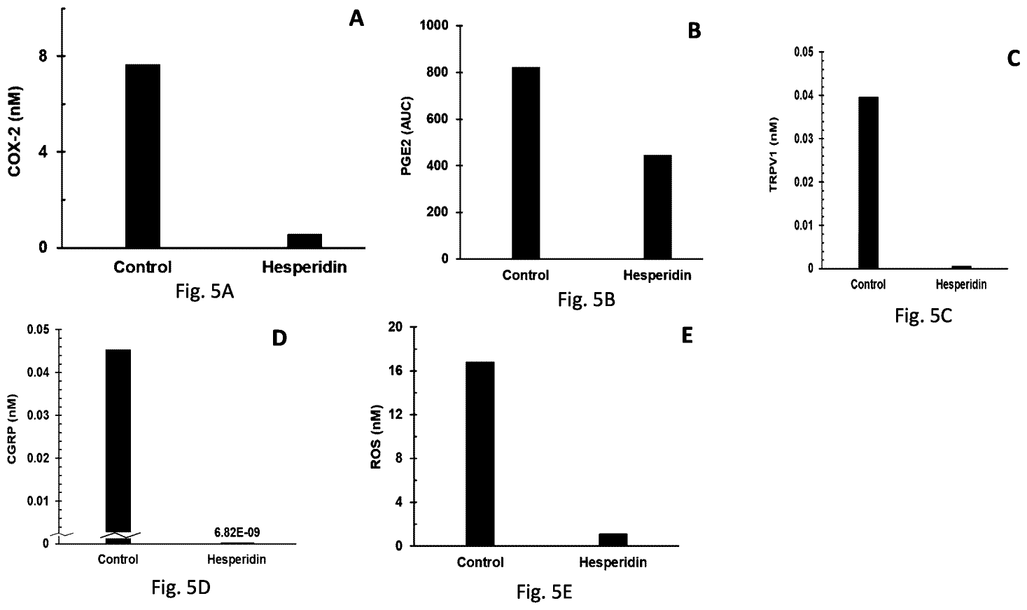

One set of outputs from Pathway Modeling Module 10 in FIG. 1 for the individual compound hesperidin 6 is its effect on the five biomarkers of joint pain 11a, as in FIG. 3. When the input to the Pathway Modeling Module 10 was individual compound hesperidin, it lowered the biomarker COX-2 compared to control as shown in FIG. 5A. When the input to the Pathway Modeling Module 10 was individual compound hesperidin, it lowered the biomarker PGE2 compared to control as shown in FIG. 5B. When the input to the Pathway Modeling Module 10 was individual compound hesperidin, it lowered the biomarker TRPV1 compared to control as shown in FIG. 5C. When the input to the Pathway Modeling Module 10 was individual compound hesperidin, it lowered the biomarker CGRP compared to control as shown in FIG. 5D. When the input to the Pathway Modeling Module 10 was individual compound hesperidin, it lowered the biomarker ROS compared to control as shown in FIG. 5E.

Sequential Compound Outputs

One set of outputs from Pathway Modeling Module 10 in FIG. 1 for the sequential addition of apigenin followed by hesperidin and hesperidin followed by apigenin 7 is their effect on the five biomarkers of joint pain 11b, as in FIG. 3.

When the input to the Pathway Modeling Module 10 was sequential addition of apigenin followed by hesperidin and hesperidin followed by apigenin, COX-2 levels are reduced, as shown in Table 1.

TABLE 1
COX-2 estimations for additive effect range of apigenin and hesperidin.

| | COX-2 |
|---|---|
| In Silico Experiment | (nM) |
| Apigenin First, Hesperidin Second | 1.93 |
| Hesperidin First, Apigenin Second | 1.54 |
| Apigenin and Hesperidin Simultaneously | 0.28 |

When the input to the System was sequential addition of apigenin followed by hesperidin and hesperidin followed by apigenin, PGE2 levels are reduced, as shown in Table 2.

TABLE 2
PGE-2 estimations for additive effects of apigenin and hesperidin.

| | PGE2 |
|---|---|
| In Silico Experiment | (AUC) |
| Apigenin First, Hesperidin Second | 289 |
| Hesperidin First, Apigenin Second | 217 |
| Apigenin and Hesperidin Simultaneously | 286 |

When the input to the System was sequential addition of apigenin followed by hesperidin and hesperidin followed by apigenin, TRPV1 levels are reduced, as shown in Table 3.

TABLE 3
TRPV1 estimations for additive effects of apigenin and hesperidin.

| | TRPV1 |
|---|---|
| In Silico Experiment | (nM) |
| Apigenin First, Hesperidin Second | 0.0027 |
| Hesperidin First, Apigenin Second | 0.0325 |
| Apigenin and Hesperidin Simultaneously | 0.00019 |

When the input to the System was sequential addition of apigenin followed by hesperidin and hesperidin followed by apigenin, CGRP levels are reduced, as shown in Table 4.

TABLE 4
CGRP estimations for additive effects of apigenin and hesperidin.

| In Silico Experiment | CGRP (nM) |
|---|---|
| Apigenin First, Hesperidin Second | 1.17 × 10⁻⁶ |
| Hesperidin First, Apigenin Second | 0.066 |
| Apigenin and Hesperidin Simultaneously | 2.65 × 10⁻¹⁰ |

When the input to the System was sequential addition of apigenin followed by hesperidin and hesperidin followed by apigenin, ROS levels are reduced, as shown in Table 5.

TABLE 5
ROS estimations for additive effects of apigenin and hesperidin.

| In Silico Experiment | ROS (nM) |
|---|---|
| Apigenin First, Hesperidin Second | 1.34 |
| Hesperidin First, Apigenin Second | 12.69 |
| Apigenin and Hesperidin Simultaneously | 1.117 |

Combination Compound Outputs

One set of outputs from Pathway Modeling Module 10 in FIG. 1 for the simultaneous addition of apigenin and hesperidin 8 is their effect on the five biomarkers of joint pain 11c, as in FIG. 3.

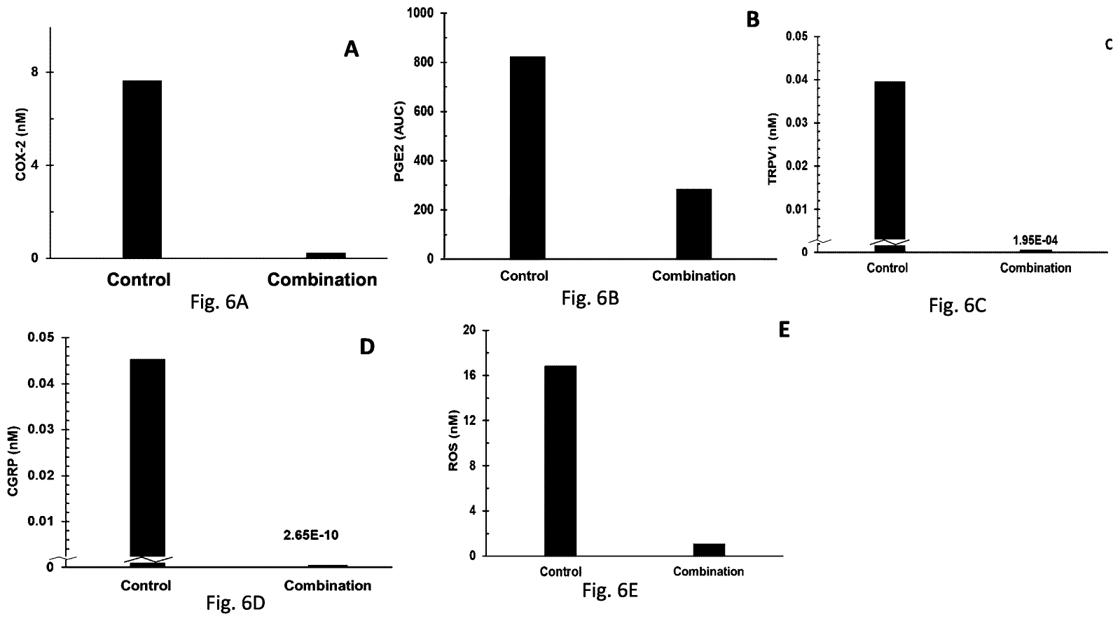

When the input to the Pathway Modeling Module 10 was simultaneous addition of apigenin and hesperidin, COX-2 levels are reduced, as shown in FIG. 6A; PGE2 levels are reduced, as shown in FIG. 6B; TRPV1 levels are reduced, as shown in FIG. 6C; CGRP levels are reduced, as shown in FIG. 6D; and, ROS levels are reduced, as shown in FIG. 6E.

Comparison Module Outputs

The Comparison Module 12 determined that combination of apigenin and hesperidin had “Synergistic Effect” on COX-2 since the COX-2 value for apigenin/hesperidin combination effect was within the additive range, as shown in Table 1.

The Comparison Module 12 determined that combination of apigenin and hesperidin had “Additive Effect” on PGE2 since the PGE2 value for apigenin/hesperidin combination effect was within the additive range, as shown in Table 2.

The Comparison Module 12 determined that combination of apigenin and hesperidin had “Synergistic Effect” on TRPV1 since the TRPV1 value for apigenin/hesperidin combination effect was within the additive range, as shown in Table 3.

The Comparison Module 12 determined that combination of apigenin and hesperidin had “Synergistic Effect” on CGRP since the CGRP value for apigenin/hesperidin combination effect was within the additive range, as shown in Table 4.

The Comparison Module 12 determined that combination of apigenin and hesperidin had “Synergistic Effect” on ROS since the ROS value for apigenin/hesperidin combination effect was within the additive range, as shown in Table 5.

Based on the Comparison Module output result of “Synergistic Effect” of apigenin and hesperidin on four biomarkers—COX-2, TRPV1, CGRP, and ROS—the System quantified the dosage for the Synergistic Combination Formula 14 in FIG. 1 for apigenin and hesperidin at a ratio of 30:1000. The apigenin/hesperidin combination at this ratio is synergistic, unexpected, and non-obvious and has been described in issued U.S. Pat. No. 11,642,360, which was filed on Sep. 3, 2020, and which is incorporated herein by reference in its entirety.

The dosage of Synergistic Combination Formula 14 in FIG. 1 for apigenin and hesperidin at 30:100 ratio was selected as a synergistic combination formula to manufacture 16 from the user interface 1 and was provided to Compound Combination System 17, which selected the Raw Material of Compounds 18 from the Synergistic Combination Formula and instructed the Manufacturing Device 19 to make the Manufactured Combination Product 16. The Manufactured Combination Product 16 is currently being distributed as mV25@.

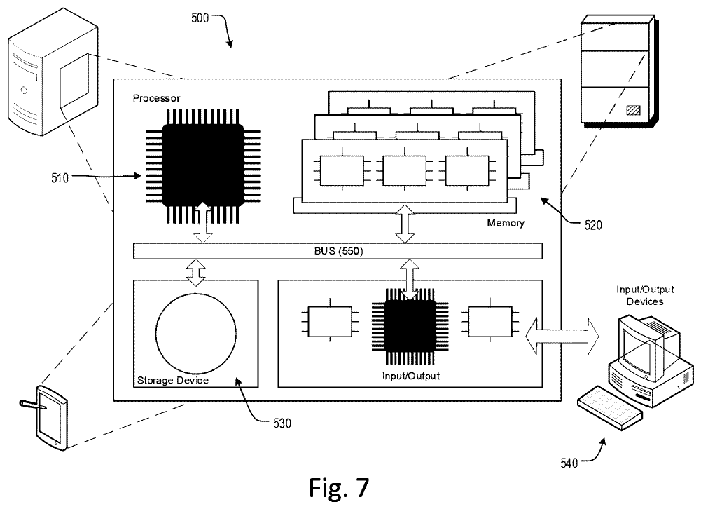

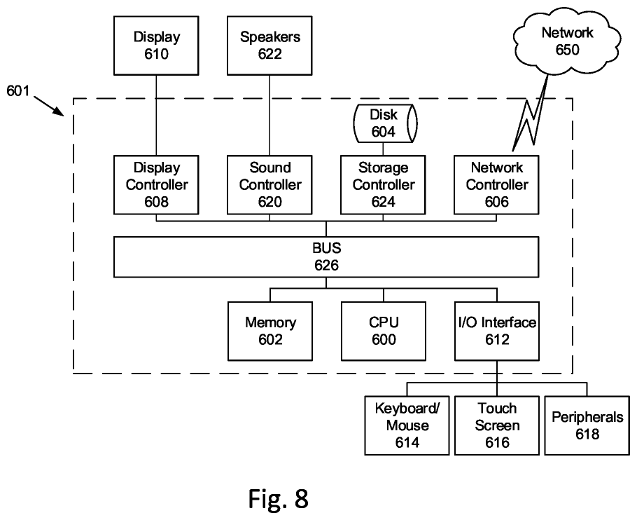

Embodiments of the subject matter and the functional operations described in this specification can be implemented by digital electronic circuitry, in tangibly embodied computer software or firmware, in computer hardware, including the structures disclosed in this specification and their structural equivalents, or in combinations of one or more of them. For example, the system illustrated in FIG. 1 can be implemented in the tangibly embodied computer software, firmware, or hardware of a single device or more than one device. Embodiments of the subject matter described in this specification can be implemented as one or more computer programs, i.e., one or more modules of computer program instructions encoded on a tangible non-transitory program carrier for execution by, or to control the operation of a data processing apparatus, such as the devices described herein with reference to FIGS. 7 and 8. The computer storage medium can be a machine-readable storage device, a machine-readable storage substrate, a random or serial access memory device, or a combination of one or more of them.

Each of the functions of the described embodiments can be implemented by one or more processing circuits/processing circuitry (may also be referred to as a controller). A processing circuit includes a programmed processor (for example, a CPU of FIG. 8), as a processor includes circuitry. A processing circuit can also include devices such as an application specific integrated circuit (ASIC) and circuit components arranged to perform the recited functions.

The term “data processing apparatus” refers to data processing hardware and may encompass all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA an ASIC.

Computers suitable for the execution of a computer program include, by way of example, general or special purpose microprocessors or both, or any other kind of central processing unit. Generally, a CPU will receive instructions and data from a read-only memory or a random access memory or both. Elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients (user devices) and servers. A client and server are generally remote from each other and typically interact through a communication network. The relationship of client and server arises by virtue of computer programs running on the respective computers and having a client-server relationship to each other. In an embodiment, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

An example of a type of computer is shown in FIG. 7. The computer 500 can be used for the operations described in association with any of the computer-implement methods described previously, according to one implementation. For example, the computer 500 can be an example of a central computing device such as the security server 100 including processing circuitry, as discussed herein. The computer 500 can be an example of any of the devices 105, 106, the commissioner device 150, the manufacturer device 160, or the networked devices 110, 111, 11n in communication with the security server 100 via a communication network. The processing circuitry includes one or more of the elements discussed next with reference to FIG. 7. In FIG. 7, the computer 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the computer 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the computer 500. In one implementation, the storage device 530 is a computer-readable medium. In various different implementations, the storage device 530 may be a floppy disk device, a hard disk device, an optical disk device, or a tape device.

The input/output device 540 provides input/output operations for the computer 500. In one implementation, the input/output device 540 includes a keyboard and/or pointing device. In another implementation, the input/output device 540 includes a display unit for displaying graphical user interfaces.

Next, a hardware description of a device 601 according to an embodiment is described with reference to FIG. 8. In FIG. 8, the device 601, which can be any of the above-described devices, including the security server 100, any of the networked devices 110, 111, 11n, the devices 105, 106, the manufacturing device 160, includes processing circuitry. The processing circuitry includes one or more of the elements discussed next with reference to FIG. 8. The process data and instructions may be stored in memory 602. These processes and instructions may also be stored on a storage medium disk 604 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 601 communicates, such as a server or computer.

Further, the claimed advancements may be provided as a utility application, background daemon, or component of an operating system, or combination thereof, executing in conjunction with CPU 600 and an operating system such as Microsoft Windows, UNIX, Solaris, LINUX, Apple MAC-OS and other systems known to those skilled in the art.

The hardware elements in order to achieve the device 601 may be realized by various circuitry elements, known to those skilled in the art. For example, CPU 600 may be a Xenon or Core processor from Intel of America or an Opteron processor from AMD of America, or may be other processor types that would be recognized by one of ordinary skill in the art. Alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described above.

The device 601 in FIG. 8 also includes a network controller 606, such as an Intel Ethernet PRO network interface card from Intel Corporation of America, for interfacing with network 650. and to communicate with the other devices. As can be appreciated, the network 650 can be a public network, such as the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 601 further includes a display controller 608, such as a NVIDIA GeForce GTX or Quadro graphics adaptor from NVIDIA Corporation of America for interfacing with display 610, such as an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners.

A sound controller 620 is also provided in the device 601 to interface with speakers/microphone 622 thereby providing sounds and/or music.

The general purpose storage controller 624 connects the storage medium disk 604 with communication bus 626, which may be an ISA, EISA, VESA, PCI, or similar, for interconnecting all of the components of the device 601. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Embodiments of the present disclosure may also be set forth in the following parentheticals.

(1) A method of determining particular combinations of compounds having a synergistic effect on a particular biological system of molecular mechanisms, the method comprising:
(a) identifying a set of N compounds, each compound of the set of N compounds interacting with a biological system of molecular mechanisms, wherein N is an integer greater than or equal to 2;
(b) determining one or more combinations of compounds, each combination of compounds consisting of two or more compounds of the set of N compounds;
(c) determining, for each combination of the one or more combinations of compounds, a set of compound sequences, each compound sequence including all compounds in the combination and being different from other of the compound sequences in the set of compound sequences;
(d) determining, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels;
(e) determining a maximum level of the set of levels as a first level of the first biomarker and a minimum level of the set of levels as a second level of the first biomarker;
(f) determining, using the mathematical model and for the particular combination, a third level of the first biomarker resulting from a simulated administration to the biological system of the compounds of the particular combination simultaneously using the first set of dosages; and
(g) determining, based on the first level, the second level, and the third level, an effect of the particular combination on the first biomarker using the first set of dosages.

(2) The method of (1), further comprising repeating steps (d) through (g) for each of the one or more combinations of compounds determined in step (b).

(3) The method of (1), further comprising determining, using the mathematical model, a set of fourth levels of the first biomarker resulting from a simulated administration to the biological system of each of the set of N compounds individually using the first set of dosages, and
determining that the effect of the particular combination is synergistic when the determined third level of the first biomarker falls outside of a range formed by the determined first level of the first biomarker and the determined second level of the first biomarker, and the determined third level of the first biomarker is greater than each of the determined fourth levels of the first biomarker.

(4) The method of (3), wherein the step of determining the effect further comprises determining that the effect of the particular combination is synergistic when determining that the third level of the first biomarker is different from a control level of the first biomarker without administration of compounds.

(5) The method of (3), further comprising outputting the first set of dosages and the two or more compounds of the particular combination when determining that the effect of the particular combination is synergistic.

(6) The method of (1), wherein
N is equal to 2, and
a first compound sequence of the set of compound sequences is a reversal of a second compound sequence of the set of compound sequences.

(7) The method of (1), further comprising receiving an input range of dosages for each compound of the particular combination and selecting the first set of dosages from within the received input range of dosages for each compound of the particular combination.

(8) The method of (7), further comprising selecting a second set of dosages from within the input range of dosages, and determining the effect of the particular combination on the first biomarker using the second set of dosages by repeating steps (d) through (g) using the second set of dosages.

(9) The method of (1), further comprising:
determining, for the particular combination, a first level, a second level, and a third level of a second biomarker of the biological system by repeating steps (d) through (f) for the second biomarker; and
determining an effect of the particular combination on the second biomarker based on the first level, the second level, and the third level of the second biomarker.

(10) The method of (1), wherein the biological system is a joint pain system, and a level of the first biomarker is indicative of joint inflammation.

(11) The method of (1), wherein the mathematical model is personalized based on a body mass index (BMI), blood test data, whole genome data, specific genes of interest for a disease or biological function, and/or gene expression data of a particular patient.

(12) The method of (1), wherein a simulation period of each simulated administration to the biological system is approximately two days.

(13) The method of (3), further comprising selecting a certain combination of compounds having the synergistic effect to be manufactured, and automatically instructing a manufacturing device to generate a product based on the selected certain combination of compounds and a corresponding set of dosages.

(14) An apparatus for determining particular combinations of compounds having a synergistic effect on a particular biological system of molecular mechanisms, the apparatus comprising:
processing circuitry configured to
   (a) identify a set of N compounds, each compound of the set of N compounds interacting with a biological system of molecular mechanisms, wherein N is an integer greater than or equal to 2;
   (b) determine one or more combinations of compounds, each combination of compounds consisting of two or more compounds of the set of N compounds;
   (c) determine, for each combination of the one or more combinations of compounds, a set of compound sequences, each compound sequence including all compounds in the combination and being different from other of the compound sequences in the set of compound sequences;
   (d) determine, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels;
   (e) determine a maximum level of the set of levels as a first level of the first biomarker and a minimum level of the set of levels as a second level of the first biomarker;
   (f) determine, using the mathematical model and for the particular combination, a third level of the first biomarker resulting from a simulated administration to the biological system of the compounds of the particular combination simultaneously using the first set of dosages; and
   (g) determine, based on the first level, the second level, and the third level, an effect of the particular combination on the first biomarker using the first set of dosages.

(15) The apparatus of (14), wherein the processing circuitry is further configured to repeat steps (d) through (g) for each of the one or more combinations of compounds determined in step (b).

(16) The apparatus of (14), wherein the processing circuitry is further configured to:
determine, using the mathematical model, a set of fourth levels of the first biomarker resulting from a simulated administration to the biological system of each of the set of N compounds individually, and
determined that the effect of the particular combination is synergistic when the determined third level of the first biomarker falls outside of a range formed by the determined first level of the first biomarker and the determined second level of the first biomarker, and the determined third level of the first biomarker is greater each of the fourth levels of the first biomarker.

(17) The apparatus of (16), wherein the processing circuitry is further configured to determine that the effect of the particular combination is synergistic when determining that the third level of the first biomarker is different from a control level of the first biomarker without administration of compounds.

(18) The apparatus of (16), wherein the processing circuitry is further configured to output the first set of dosages and the two or more compounds of the particular combination when determining that the effect of the particular combination is synergistic.

(19) A system for determining particular combinations of compounds having a synergistic effect on a particular biological system of molecular mechanisms, the system comprising:
a biological system modeling device having first processing circuitry;
a computer-readable memory; and
a manufacturing device having second processing circuitry,
wherein the first processing circuitry is configured to
   (a) identify a set of N compounds, each compound of the set of N compounds interacting with a biological system of molecular mechanisms, wherein N is an integer greater than or equal to 2;
   (b) determine one or more combinations of compounds, each combination of compounds consisting of two or more compounds of the set of N compounds;
   (c) determine, for each combination of the one or more combinations of compounds, a set of compound sequences, each compound sequence including all compounds in the combination and being different from other of the compound sequences in the set of compound sequences;
   (d) determine, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels;
   (e) determine a maximum level of the set of levels as a first level of the first biomarker and a minimum level of the set of levels as a second level of the first biomarker;
   (f) determine, using the mathematical model and for the particular combination, a third level of the first biomarker resulting from a simulated administration to the biological system of the compounds of the particular combination simultaneously using the first set of dosages;
   (g) determine, based on the first level, the second level, and the third level, an effect of the particular combination on the first biomarker using the first set of dosages; and
   (h) store, when determining that the effect of the particular combination on the first biomarker is synergistic, the particular combination and the first set of dosages in the computer-readable memory, and
wherein the second processing circuitry is configured to
   access the particular combination and the first set of dosages stored in the computer-readable memory, and
   generate a product based on the particular combination and the first set of dosages.

(20) The system of (19), wherein the first processing circuitry is further configured to
determine, using the mathematical model, a set of fourth levels of the first biomarker resulting from a simulated administration to the biological system of each of the set of N compounds individually, and
determine that the effect of the particular combination is synergistic when the determined third level of the first biomarker falls outside of a range formed by the determined first level of the first biomarker and the determined second level of the first biomarker, and the determined third level of the first biomarker is greater than each of the determined fourth levels of the first biomarker.

Numerous modifications and variations of the present disclosure are possible in light of the above teachings. It is therefore to be understood that within the scope of the appended claims, the disclosure may be practiced otherwise than as specifically described herein.

1. A method of determining particular combinations of compounds having a synergistic effect on a particular biological system of molecular mechanisms, the method comprising:
(a) identifying a set of N compounds, each compound of the set of N compounds interacting with a biological system of molecular mechanisms, wherein N is an integer greater than or equal to 2;
(b) determining one or more combinations of compounds, each combination of compounds consisting of two or more compounds of the set of N compounds;
(c) determining, for each combination of the one or more combinations of compounds, a set of compound sequences, each compound sequence including all compounds in the combination and being different from other of the compound sequences in the set of compound sequences;
(d) determining, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels;
(e) determining a maximum level of the set of levels as a first level of the first biomarker and a minimum level of the set of levels as a second level of the first biomarker;
(f) determining, using the mathematical model and for the particular combination, a third level of the first biomarker resulting from a simulated administration to the biological system of the compounds of the particular combination simultaneously using the first set of dosages; and
(g) determining, based on the first level, the second level, and the third level, an effect of the particular combination on the first biomarker using the first set of dosages.

2. The method of claim 1, further comprising repeating steps (d) through (g) for each of the one or more combinations of compounds determined in step (b).

3. The method of claim 1, further comprising determining, using the mathematical model, a set of fourth levels of the first biomarker resulting from a simulated administration to the biological system of each of the set of N compounds individually using the first set of dosages, and
determining that the effect of the particular combination is synergistic when the determined third level of the first biomarker falls outside of a range formed by the determined first level of the first biomarker and the determined second level of the first biomarker, and the determined third level of the first biomarker is greater than each of the determined fourth levels of the first biomarker.

4. The method of claim 3, wherein the step of determining the effect further comprises determining that the effect of the particular combination is synergistic when determining that the third level of the first biomarker is different from a control level of the first biomarker without administration of compounds.

5. The method of claim 3, further comprising outputting the first set of dosages and the two or more compounds of the particular combination when determining that the effect of the particular combination is synergistic.

6. The method of claim 1, wherein
N is equal to 2, and
a first compound sequence of the set of compound sequences is a reversal of a second compound sequence of the set of compound sequences.

7. The method of claim 1, further comprising receiving an input range of dosages for each compound of the particular combination and selecting the first set of dosages from within the received input range of dosages for each compound of the particular combination.

8. The method of claim 7, further comprising selecting a second set of dosages from within the input range of dosages, and determining the effect of the particular combination on the first biomarker using the second set of dosages by repeating steps (d) through (g) using the second set of dosages.

9. The method of claim 1, further comprising:
determining, for the particular combination, a first level, a second level, and a third level of a second biomarker of the biological system by repeating steps (d) through (f) for the second biomarker; and
determining an effect of the particular combination on the second biomarker based on the first level, the second level, and the third level of the second biomarker.

10. The method of claim 1, wherein the biological system is a joint pain system, and a level of the first biomarker is indicative of joint inflammation.

11. The method of claim 1, wherein the mathematical model is personalized based on a body mass index (BMI), blood test data, whole genome data, specific genes of interest for a disease or biological function, and/or gene expression data of a particular patient.

12. The method of claim 1, wherein a simulation period of each simulated administration to the biological system is approximately two days.

13. The method of claim 3, further comprising selecting a certain combination of compounds having the synergistic effect to be manufactured, and automatically instructing a manufacturing device to generate a product based on the selected certain combination of compounds and a corresponding set of dosages.

14. An apparatus for determining particular combinations of compounds having a synergistic effect on a particular biological system of molecular mechanisms, the apparatus comprising:
processing circuitry configured to
(a) identify a set of N compounds, each compound of the set of N compounds interacting with a biological system of molecular mechanisms, wherein N is an integer greater than or equal to 2;
(b) determine one or more combinations of compounds, each combination of compounds consisting of two or more compounds of the set of N compounds;
(c) determine, for each combination of the one or more combinations of compounds, a set of compound sequences, each compound sequence including all compounds in the combination and being different from other of the compound sequences in the set of compound sequences;
(d) determine, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels;
(e) determine a maximum level of the set of levels as a first level of the first biomarker and a minimum level of the set of levels as a second level of the first biomarker;
(f) determine, using the mathematical model and for the particular combination, a third level of the first biomarker resulting from a simulated administration to the biological system of the compounds of the particular combination simultaneously using the first set of dosages; and
(g) determine, based on the first level, the second level, and the third level, an effect of the particular combination on the first biomarker using the first set of dosages.

15. The apparatus of claim 14, wherein the processing circuitry is further configured to repeat steps (d) through (g) for each of the one or more combinations of compounds determined in step (b).

16. The apparatus of claim 14, wherein the processing circuitry is further configured to:
determine, using the mathematical model, a set of fourth levels of the first biomarker resulting from a simulated administration to the biological system of each of the set of N compounds individually, and
determined that the effect of the particular combination is synergistic when the determined third level of the first biomarker falls outside of a range formed by the determined first level of the first biomarker and the determined second level of the first biomarker, and the determined third level of the first biomarker is greater each of the fourth levels of the first biomarker.

17. The apparatus of claim 16, wherein the processing circuitry is further configured to determine that the effect of the particular combination is synergistic when determining that the third level of the first biomarker is different from a control level of the first biomarker without administration of compounds.

18. The apparatus of claim 16, wherein the processing circuitry is further configured to output the first set of dosages and the two or more compounds of the particular combination when determining that the effect of the particular combination is synergistic.

19. A system for determining particular combinations of compounds having a synergistic effect on a particular biological system of molecular mechanisms, the system comprising:
a biological system modeling device having first processing circuitry;
a computer-readable memory; and
a manufacturing device having second processing circuitry,
wherein the first processing circuitry is configured to
(a) identify a set of N compounds, each compound of the set of N compounds interacting with a biological system of molecular mechanisms, wherein N is an integer greater than or equal to 2;
(b) determine one or more combinations of compounds, each combination of compounds consisting of two or more compounds of the set of N compounds;
(c) determine, for each combination of the one or more combinations of compounds, a set of compound sequences, each compound sequence including all compounds in the combination and being different from other of the compound sequences in the set of compound sequences;
(d) determine, using a mathematical model that describes the biological system and for each compound sequence in the determined set of compound sequences of a particular combination of the one or more combinations, a level of a first biomarker of the biological system resulting from a simulated administration to the biological system of the compound sequence sequentially in time using a first set of dosages, so as to obtain a set of levels;
(e) determine a maximum level of the set of levels as a first level of the first biomarker and a minimum level of the set of levels as a second level of the first biomarker;
(f) determine, using the mathematical model and for the particular combination, a third level of the first biomarker resulting from a simulated administration to the biological system of the compounds of the particular combination simultaneously using the first set of dosages;
(g) determine, based on the first level, the second level, and the third level, an effect of the particular combination on the first biomarker using the first set of dosages; and
(h) store, when determining that the effect of the particular combination on the first biomarker is synergistic, the particular combination and the first set of dosages in the computer-readable memory, and
wherein the second processing circuitry is configured to
access the particular combination and the first set of dosages stored in the computer-readable memory, and
generate a product based on the particular combination and the first set of dosages.

20. The system of claim 19, wherein the first processing circuitry is further configured to
determine, using the mathematical model, a set of fourth levels of the first biomarker resulting from a simulated administration to the biological system of each of the set of N compounds individually, and
determine that the effect of the particular combination is synergistic when the determined third level of the first biomarker falls outside of a range formed by the determined first level of the first biomarker and the determined second level of the first biomarker, and the determined third level of the first biomarker is greater than each of the determined fourth levels of the first biomarker.
